(19) United States
(12) Patent Application Publication
(10) Pub. No.: US 2010/0254853 A1
(43) Pub. Date: Oct. 7, 2010

(54) Method of sterilization using plasma generated sterilant gas

(76) Inventors: Sang Hun Lee, San Ramon, CA (US); Joong Soo Kim, Los Altos, CA (US); Jeo-Mo Koo, Palo Alto, CA (US); Andrew Way, Sunnyvale, CA (US); Orion Weihe, Fremont, CA (US); Jeff Ifland, Cupertino, CA (US)

Correspondence Address: JORDAN AND HAMBURG LLP, 122 EAST 42ND STREET, SUITE 4000, NEW YORK, NY, 10168, US

(21) Appl. No.: 12/386,578

(22) Filed: Apr. 21, 2009

Related U.S. Application Data
(63) Continuation-in-part of application No. 12/384,536, filed on Apr. 6, 2009.

(51) Int. Cl. A61L 9/015; B01J 19/08

(52) U.S. Cl. 422/29; 422/186.21

(57) ABSTRACT

A sterilizing apparatus and method provide a sterilizing system having a sterilization chamber and a method for sterilizing an item in the sterilization chamber. The method includes the steps of: loading the item into the sterilization chamber; evacuating gas from the sterilization chamber; preparing sterilant gas by use of plasma; and filling the sterilization chamber with the sterilant gas to a preset pressure. The method further includes the steps of waiting a preset time interval to thereby accomplish an intended sterilization and evacuating the sterilant gas from the sterilization chamber.

### CROSS REFERENCE TO RELATED APPLICATIONS

This application is a continuation-in-part of application Ser. No. 12/384,536, filed on Apr. 6, 2009, entitled “STERILANT GAS GENERATING SYSTEM,” invented by Sang H. Lee et al., having attorney docket number F-9868.

### BACKGROUND OF THE INVENTION

1. Field of the Invention

The present invention relates to sterilization, and more particularly to methods of sterilization using plasma generated sterilant gas.

2. Discussion of the Related Art

Steam autoclaving is the most commonly accepted standard for sterilizing most medical instruments. During sterilization, the instruments are exposed to steam at 121° C. at 15-20 lbs of pressure for 15-30 minutes. One of the disadvantages of autoclaving method is that this method is not suitable for plastics and other heat labile materials.

As an alternative, various sterilant gases, such as nitric oxide, nitrogen dioxide, sulfur dioxide, hydrogen peroxide, chlorine dioxide, carbon dioxide, ozone, and ethylene oxide, have been used to kill or control the growth of microbial contaminations. In conventional systems, generating and handling these sterilant gases in high concentrations may represent hazard to the human operators, which may impose a limit on the allowable concentration of gas unless an effective approach to resolve this safety issue is provided. It is because if the concentration of the sterilant gas needs be decreased due to safety concerns, the exposure time required to complete a sterilization process must be increased. Thus, there is a need for methods and devices that can generate sterilant gases of high concentration in a safe and efficient manner so that the potential hazard to human operators can be minimized.

### SUMMARY OF THE INVENTION

According to one aspect of the present invention, a method for sterilizing an item includes the steps of: (a) loading the item in a sterilization chamber; (b) preparing sterilant gas by use of a plasma; and (c) filling the sterilization chamber with the sterilant gas to a preset pressure to form a gas mixture.

According to another aspect of the present invention, an apparatus for sterilizing an item includes: a sterilization chamber for loading the item therein; a plasma generator for generating a plasma that produces sterilant gas; and a controller adapted to fill the sterilization chamber with the sterilant gas to a preset pressure.

According to yet another aspect of the present invention, a system for sterilizing a target includes: a chamber having a space for loading a target therein; and a sterilant gas supply for producing sterilant gas by use of a plasma and providing the sterilant gas to the chamber.

The above, and other objects, features and advantages of the present invention will become apparent from the following description read in conjunction with the accompanying drawings, in which like reference numerals designate the same elements. The present invention is considered to include all functional combinations of the above described features and is not limited to the particular structural embodiments shown in the figures as examples. The scope and spirit of the present invention is considered to include modifications as may be made by those skilled in the art having the benefit of the present disclosure which substitute, for elements or processes presented in the claims, devices or structures or processes upon which the claim language reads or which are equivalent thereto, and which produce substantially the same results associated with those corresponding examples identified in this disclosure for purposes of the operation of this invention. Additionally, the scope and spirit of the present invention is intended to be defined by the scope of the claim language itself and equivalents thereto without incorporation of structural or functional limitations discussed in the specification which are not referred to in the claim language itself. Still further it is understood that recitation of the preface of “a” or “an” before an element of a claim does not limit the claim to a singular presence of the element and the recitation may include a plurality of the element unless the claim is expressly limited otherwise. Yet further it will be understood that recitations in the claims which do not include “means for” or “steps for” language are not to be considered limited to equivalents of specific embodiments described herein.

### BRIEF DESCRIPTION OF THE DRAWINGS

FIG. 1 shows a schematic diagram of an NO_{X} generating system in accordance with one embodiment of the present invention.

FIG. 2 shows an exploded view of a portion of the NO_{X} generating system of FIG. 1.

FIG. 3 shows a side cross-sectional view of a portion of the NO_{X} generating system of FIG. 1, taken along the line III-III.

FIG. 4 shows a schematic diagram of an NO_{X} generating system in accordance with another embodiment of the present invention.

FIG. 5 shows a schematic diagram of an NO_{X} generating system in accordance with yet another embodiment of the present invention.

FIG. 6 shows a schematic diagram of an NO_{X} generating system in accordance with still another embodiment of the present invention.

FIG. 7 shows a perspective view of a sterilization device in accordance with another embodiment of the present invention.

FIG. 8 shows a flow chart illustrating a process for sterilizing target items in accordance with another embodiment of the present invention.

### DETAILED DESCRIPTION OF THE PREFERRED EMBODIMENTS

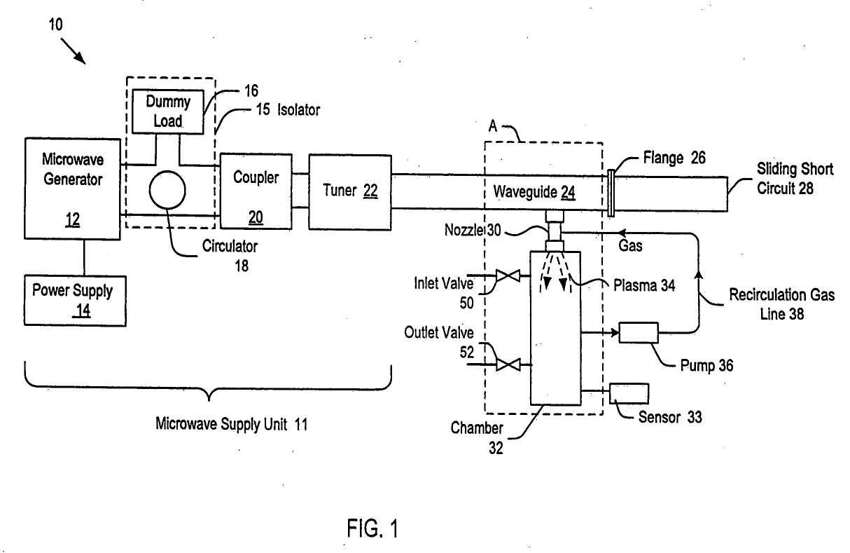

FIG. 1 shows a schematic diagram of an NO_{X} generating system 10 in accordance with one embodiment of the present invention. It is noted that the disclosed exemplary embodiments of the present invention are directed to generating and handling NO_{X}, such as NO and NO₂. However, it should be apparent to those of ordinary skill in the art that the disclosed embodiments can be used to generate and handle other types of sterilant gases (or, equivalently, target gases), such as CO₂, ClO₂, SO₂, H₂O₂, O₃, and EtO.

As depicted in FIG. 1, the system 10 includes: a microwave cavity/waveguide 24; a microwave supply unit 11 for providing microwave energy to the microwave waveguide 24; a nozzle 30 connected to the microwave waveguide 24 and operative to receive microwave energy from the microwave waveguide 24 and excite gas by use of the received microwave energy; a sliding short circuit 28 disposed at the end of the waveguide 24; a chamber 32 for receiving and containing the gas that exits the nozzle 30; a pump 36 for recirculating the NO_{X} containing gas contained in the chamber 32 via a recirculation gas line 38; a sensor 33 for measuring the NO_{X} concentration in the chamber 32; an inlet valve 50; and an outlet valve 52. The nozzle 30 may excite the gas provided via the recirculating gas line 38 into plasma 34.

The inlet valve 50 is used to fill the chamber 32 with gas including nitrogen and oxygen. Upon filling the chamber 32 to a preset pressure, the inlet valve 50 is closed. Then, the microwave supply unit 11 is operated to generate plasma at the nozzle 30 and to recirculate the gas contained in the chamber 32 so that the gas contained in the chamber 32 includes NO_{X}. It is noted that those skilled in the art will understand that the volume fractions of nitrogen and oxygen introduced in the chamber 32 via the inlet valve 50 may be varied according to the intended concentration of the target sterilant gas component contained in the chamber 32 and various types of sensors can be used to measure the concentration of the target gas component. The outlet valve 52 may be connected to another device (not shown in FIG. 1), such as sterilization chamber, that utilizes the NO_{X} gas discharged from the chamber 32 through the outlet valve 52. The inlet valve 50 and the outlet valve 52 are secured to the sidewall of the chamber 32. However, it should be apparent to those of ordinary skill that these valves can be disposed in any other suitable locations without deviating from the spirit and scope of the present teachings.

As discussed above, the system 10 can be used to generate other types of sterilant gases. For example, the system 10 can be used to generate ozone by introducing pure oxygen into the chamber 32 via the inlet valve 50. In another example, the system 10 can be used to generate chlorine dioxide by introducing a mixture of oxygen and chlorine into the chamber 32 via the inlet valve 50.

The microwave supply unit 11 provides microwave energy to the microwave waveguide 24 and includes: a microwave generator 12 for generating microwaves; a power supply 14 for supplying power to the microwave generator 12; and an isolator 15 having a dummy load 16 for dissipating reflected microwave energy that propagates toward the microwave generator 12 and a circulator 18 for directing the reflected microwave energy to the dummy load 16.

The microwave supply unit 11 may further include a coupler 20 for measuring fluxes of the microwave energy; and a tuner 22 for reducing the microwave energy reflected from the sliding short circuit 28. The components of the microwave supply unit 11 shown in FIG. 1 are listed herein for exemplary purposes only. Also, it is possible to replace the microwave supply unit 11 with any other suitable system having the capability to provide microwave energy to the microwave waveguide 24 without deviating from the spirit and scope of the present teachings. Likewise, the sliding short circuit 28 may be replaced by a phase shifter that can be configured in the microwave supply unit 11. Optionally, a phase shifter (not shown in FIG. 1) may be mounted between the isolator 15 and the coupler 20.

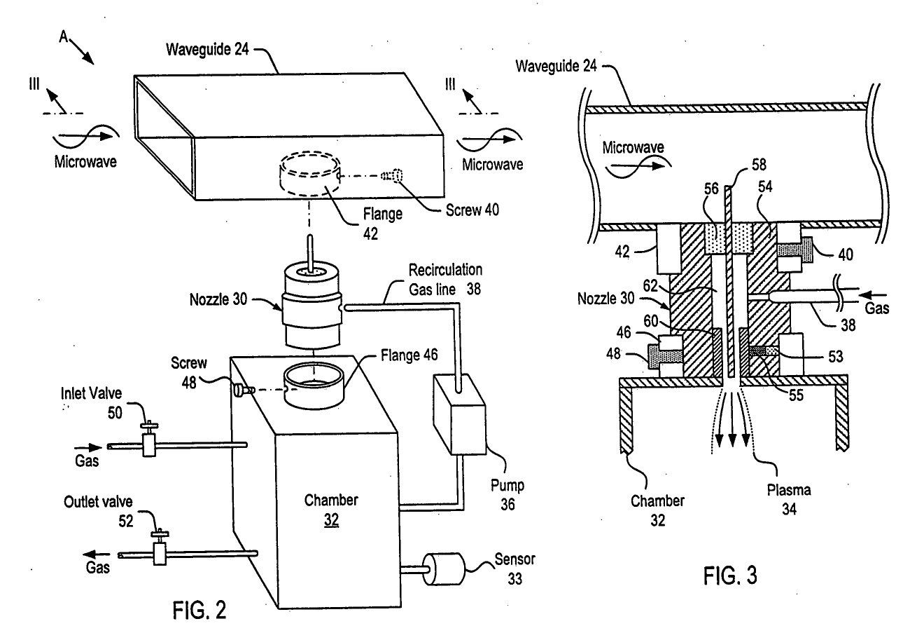

FIG. 2 shows an exploded view of a portion A of the NO_{X} generating system 10 of FIG. 1. FIG. 3 shows a side cross-sectional view of the portion A of the NO_{X} generating system 10, taken along the line III-III. As depicted, a ring-shaped flange 42 is affixed to a bottom surface of the microwave cavity 24 and the nozzle 30 is secured to the ring-shaped flange 42 by one or more suitable fasteners 40, such as screws.

The nozzle 30 includes a rod-shaped conductor 58; a housing or shield 54 formed of conducting material, such as metal, and having a generally cylindrical cavity/space 62 formed thererin so that the space forms a gas flow passageway; an electrical insulator 56 disposed in the space and adapted to hold the rod-shaped conductor 58 relative to the shield 54; a dielectric tube (such as quartz tube) 60; a spacer 55; and a fastener 53, such as a metal screw, for pushing the spacer 55 against the dielectric tube 60 to thereby secure the dielectric tube 60 to the housing 54. The spacer 55 is preferably formed of dielectric material, such as Teflon®, and functions as a buffer for firmly pushing the dielectric tube 60 against the shield 54 without cracking the dielectric tube 60.

The top portion (or, equivalently, proximal end portion) of the rod-shaped conductor 58 functions as an antenna to pick up microwave energy in the microwave cavity 24. The microwave energy captured by the rod-shaped conductor 58 flows along the surface thereof. The gas supplied via a gas line 38 is injected into the space 62 and excited by the microwave energy flowing along the surface of the rod-shaped conductor 58. Plasma 34 may be formed at the bottom tip portion (or, equivalently, distal end portion) of the rod-shaped conductor 58.

In the plasma 34, the gas including nitrogen and oxygen molecules chemically react to generate various types of gas species including NOx and free radicals. In the process of recirculating the gas contained in the chamber 32 via the recirculation gas line 38, the plasma 34 continuously generates the NOx particles and, as a consequence, the concentrations of NOx particles in the chamber 34 increase quite rapidly. Also, during the recirculation process, the recirculated NOx species and free radicals participate in the chemical reactions in the plasma 34 to thereby promote the chemical reactions. When the concentration of the NOx species in the chamber 32 reaches an intended level, the gas contained in the chamber 32 may be discharged to a device (not shown in FIGS. 1-3), such as a sterilization apparatus, via the outlet valve 52.

A ring-shaped flange 46 is affixed to the top surface of the chamber 32 and the nozzle 30 is secured to the ring-shaped flange 46 by one or more suitable fasteners 48, such as screws. It is noted that the nozzle 30 may be secured to the chamber 32 by any other suitable types of securing mechanisms.

The rod-shaped conductor 58, the dielectric tube 60, and the electric insulator 56 have functions similar to those of their counterparts of a nozzle described in U.S. Pat. No. 7,164,095, which is herein incorporated by reference in its entirety. For brevity, these components are not described in detail in the present document.

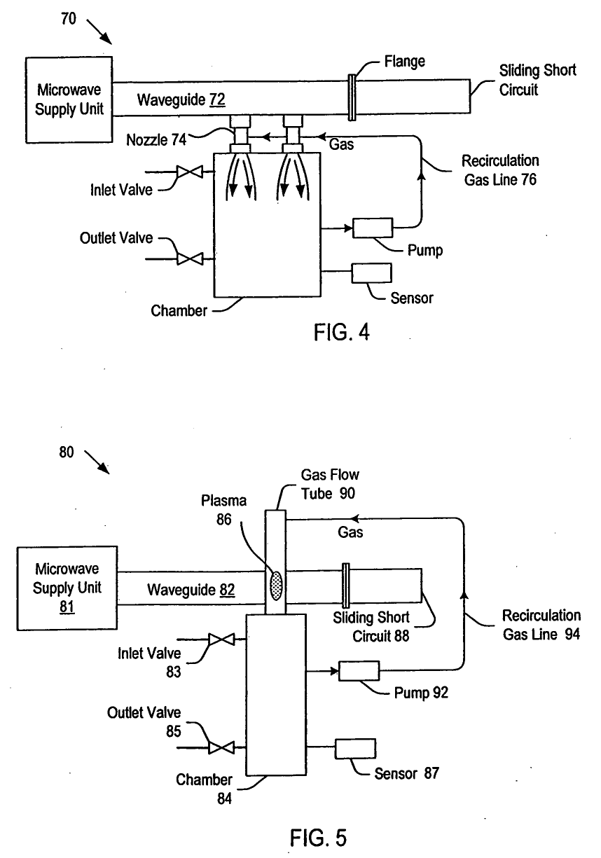

FIG. 4 shows a schematic diagram of a NOx generating system 70 in accordance with another embodiment of the present invention in which like part parts are configured similar to those of the above embodiment except for as set forth below. As depicted, the system 70 is similar to the system 10, with the difference in the number of nozzles 74 attached to the waveguide 72. The nozzle 74 may be similar to the nozzle 30 in FIGS. 1-3. The recirculation gas line 76 has one or more manifolds (not shown in FIG. 4) to provide the recirculated gas to the nozzles 74.

In the nozzles 30, 74, the threshold intensity of the microwave energy required to ignite plasma can be controlled if the point where the microwave energy is focused can be moved relative to the nozzle exit. Typically, the microwave energy is focused at the bottom tip portion of the rod-shaped conductor. Thus, to control the plasma ignition, a mechanism to move the rod-shaped conductor relative to the nozzle housing optionally can be installed in each of the nozzles 30, 74, and may be implemented based on this direction in various ways by those skilled in the art. More detailed information of a mechanism to move the rod-shaped conductor can be found in U.S. patent application Ser. No. 12/291,646, entitled “Plasma generating system having tunable plasma nozzle,” filed on Nov. 12, 2008, which is herein incorporated by reference in its entirety. For brevity, a nozzle having a mechanism to move the rod-shaped conductor similar to the mechanism described in the copending U.S. patent application Ser. No. 12/291,646 is not shown in the present document as not necessary for the practice of the present invention.

FIG. 5 shows a schematic diagram of a NOx generating system 80 in accordance with yet another embodiment of the present invention in which like part parts are configured similar to those of the above embodiments except for as set forth below. As depicted, the system 80 includes: a microwave cavity/waveguide 82; a microwave supply unit 81 for providing microwave energy to the microwave waveguide 82; a gas flow tube 90 extending through the waveguide 82; a chamber 84 coupled to the exit of the gas flow tube 90 and adapted to receive and contain the gas that exits the gas flow tube 90; a pump 92 for recirculating the NOx containing gas contained in the chamber 84 via a recirculation gas line 94; a sensor 87 for measuring the NOx concentration in the chamber 84; an inlet valve 83; and an outlet valve 85; and, optionally, a sliding short circuit 88 disposed at the end of the waveguide 82.

The gas flow tube 90 may be formed of dielectric material, such as quartz, transparent to the microwave energy. The inlet of the gas flow tube 90 is coupled to the recirculation gas line 94. As the gas flows through the gas flow tube 90, the gas is excited by the microwave energy in the waveguide 82 and subject to chemical reactions. Depending on the intensity of the microwave energy in the waveguide 82, plasma 86 may be ignited in the gas flow tube 90.

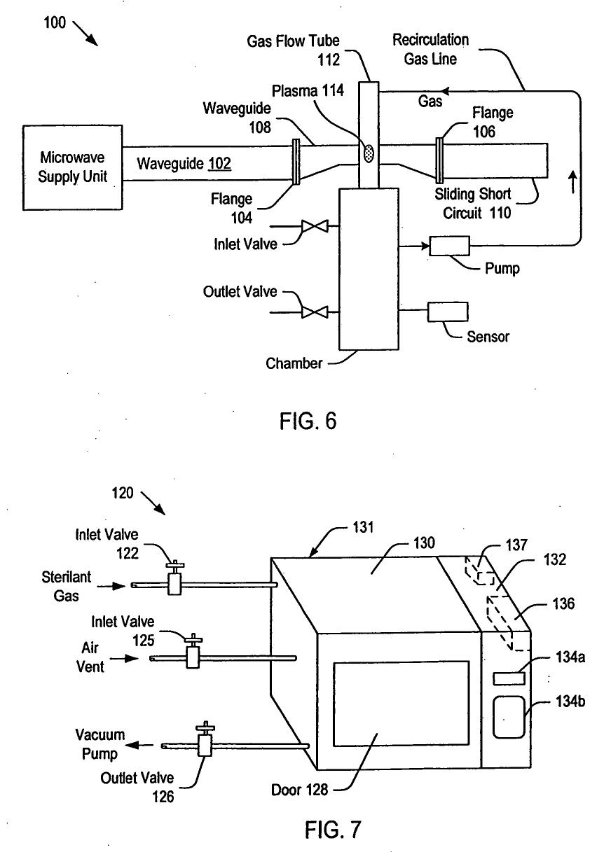

FIG. 6 shows a schematic diagram of a NOx generating system 100 in accordance with still another embodiment of the present invention in which like part parts are configured similar to those of the above embodiments except for as set forth below. As depicted, the system 100 is similar to the system 80, with the difference that an additional waveguide 108 is disposed between a waveguide 102 and a sliding short circuit 110 by use of flanges 104, 106. The cross-sectional dimension of the waveguide 108 is varied along the direction of the microwave propagation to enhance the microwave energy intensity per unit area near the location where the gas flow tube 112 passes and to thereby reduce the threshold microwave intensity required to ignite plasma 114 in the gas flow tube 112.

FIG. 7 shows a perspective view of a sterilization device 120 that might be used with the systems 10, 70, 80, and 100 in accordance with another embodiment of the present invention. As depicted, the sterilization device 120 includes: an outer enclosure 131 housing a sterilization chamber 130 and an electronic compartment 132; a first inlet valve 122 connected to the outlet valve of the systems 10, 70, 80, and 100 and configured to introduce the sterilant gas into the sterilization chamber 130 therethrough; a vent 125 for discharging the gas inside the chamber 130 or introducing the air into the chamber 130; and an outlet valve 126 connected to a vacuum pump (not shown in FIG. 7) and configured to evacuate the gas from the sterilization chamber 130. The outer enclosure 131 includes a door 128 through which target items having microorganisms to be sterilized are loaded into or unloaded from the chamber 130. The outer enclosure 131 also includes a display 134a and a user interface 134b that allow the user to control the device 120. For instance, a plurality of control buttons may be included in the user interface 134b and the display 134a may display the information input by the user. It is noted that the device 120 may have any other suitable number and types of displays and user interfaces without deviating from the spirit and scope of the present teachings.

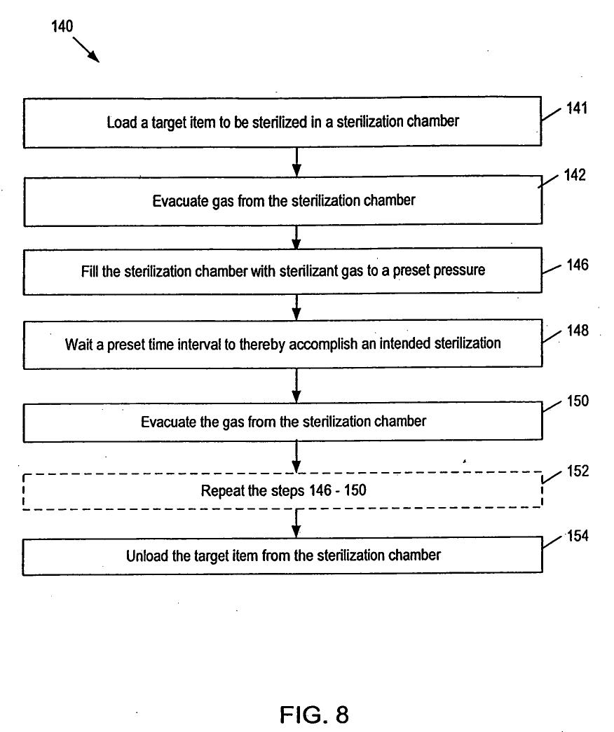

The sterilization device 120 may include an electronic controller 136 for controlling the components of the device 120. For instance, the user may program a processor included in the controller 136 so that the sterilization process described in connection with FIG. 8 may be performed as programmed by the user.

One or more sensors 137, such as a thermometer, barometer, and a sterilant gas concentration sensor, may be installed in the electronic compartment 132. These sensors 137 may be used to control the temperature, pressure, and sterilant gas concentration of the gas in the sterilization chamber 130. For instance, a sterilant gas concentration sensor, the inlet valve 122 and/or outlet valve 126, and the controller 136 may form a feedback control system to control the concentration of the sterilant gas in the chamber 130.

It is noted that the device 120 may have other components. For example, the door 128 may include a window through which the user may have a visual inspection of the target items in the chamber 130. In another example, the door 128 may have a handle (not shown in FIG. 7) for the user to unlatch and open the door 128. The latch mechanism may contain a series of mechanical switches that function as a safety interlock to inform the device 120 the door 128 is open (or not properly closed) and to de-activate the first inlet valve 122 thus preventing accidental leakage of the sterilant gas through the door 128.

FIG. 8 shows a flow chart 140 illustrating a process for sterilizing target items in the sterilization device 120 according to another embodiment of the present invention. The process may begin in a step 141. In the step 141, the user loads a target item to be sterilized in the sterilization chamber 130. Then, in a step 142, the user evacuates gas from the chamber via the outlet valve 126 by use of a vacuum pump. Next, in a step 146, the user fills the sterilization chamber 130 with sterilant gas via the first inlet valve 122 to a preset pressure. Then, in a step 148, the user waits a preset time interval for an intended sterilization to be accomplished in the chamber 130. The preset time may vary depending on various parameters, such as the types of the sterilant gas, the geometry of the targets, and the target microorganisms to be sterilized. Next, the user evacuates the gas from the chamber 130 in a step 150. Optionally, in a step 152, the steps 146-150 may be repeated until the sterilization process is completed. Finally, in a step 154, the user unloads the target item from the sterilization chamber 130. It is noted that the user may program the device 120 so that one or more of the steps 141-154 may be performed without human intervention.

Having described preferred embodiments of the invention with reference to the accompanying drawings, it is to be understood that the invention is not limited to those precise embodiments, and that various changes and modifications may be effected therein by one skilled in the art without departing from the scope or spirit of the inventions defined in the appended claims. Such modifications include substitution of components for components specifically identified herein, wherein the substitute component provides functional results which permit the overall functional operation of the present invention to be maintained. Such substitutions are intended to encompass as replacements for components and components yet to be developed which are accepted as replacements for components identified herein and which produce results compatible with operation of the present invention. Furthermore, the signals used in this invention are considered to encompass any electromagnetic wave transmission.

What is claimed is:

1. A method for sterilizing an item, comprising:
(a) loading the item into a sterilization chamber;
(b) preparing sterilant gas by use of a plasma; and
(c) filling the sterilization chamber with the sterilant gas to a preset pressure with the item loaded therein.

2. A method as recited in claim 1, further comprising:
(d) waiting a preset time interval with the item in the chamber filled with the sterilant gas to the preset level wherein the preset time interval is sufficient to accomplish an intended sterilization; and
(e) evacuating the sterilant gas from the sterilization chamber following expiration of the preset time interval.

3. A method as recited in claim 2, further comprising repeating the steps (b)-(e).

4. A method as recited in claim 2, further comprising repeating the steps (c)-(e).

5. A method as recited in claim 1, wherein the sterilant gas is NOₓ.

6. A system for sterilizing a target, comprising:
a chamber having a space for loading a target therein; and
a sterilant gas supply for producing sterilant gas by use of a plasma and providing the sterilant gas to the chamber.

7. A system as recited in claim 6, wherein the sterilant gas supply includes a plasma generator for generating the plasma that produces the sterilant gas.

8. A system as recited in claim 6, wherein the sterilant gas includes NOx species.

9. A system as recited in claim 6, further comprising a vacuum pump for evacuating gas from the chamber.

10. A system as recited in claim 6, further comprising at least one sensor for sensing at least one of a temperature, a pressure, or a concentration of the sterilant gas in the chamber.

11. An apparatus for sterilizing an item, comprising:
a sterilization chamber for loading the item therein;
a plasma generator configured to generate a plasma transforming gas into sterilant gas; and
a controller configured to fill the sterilization chamber with the sterilant gas to a preset pressure.

12. An apparatus as recited in claim 11, wherein the controller is further configured to wait a preset time interval, after the sterilization chamber is filled with the item and the sterilant gas, sufficient to accomplish an intended sterilization and to evacuate the sterilant gas from the sterilization chamber after the preset time interval has passed.

13. An apparatus as recited in claim 11, further comprising at least one sensor for sensing at least one of a temperature, a pressure, and a concentration of the sterilant gas in the sterilization chamber.
